(19) United States
(12) Patent Application Publication
(10) Pub. No.: US 2006/0265060 A1
(43) Pub. Date: Nov. 23, 2006

(54) Breast prosthesis

(75) Inventors: Nils Stelter, Achenmuchle (DE); Helmut Wild, Rosenheim (DE)

Correspondence Address: ALLEMAN HALL MCCOY RUSSELL & TUTTLE LLP, 806 SW BROADWAY, SUITE 600, PORTLAND, OR, 97205-3335, US

(73) Assignee: AMOENA Medizin-Orthopaedie-Technik GmbH

(21) Appl. No.: 11/435,465

(22) Filed: May 16, 2006

(30) Foreign Application Priority Data
May 17, 2005 (DE) 20 2005 007 677.5

(51) Int. Cl. A61F 2/52; A41C 3/10

(52) U.S. Cl. 623/7; 450/55

(57) ABSTRACT

The present disclosure relates to a breast prosthesis comprising bodies which copy the shape of the breast, and which are preferably made from an addition-crosslinking two component silicone rubber mass and which are sealed in plastic films. According the present disclosure, there is a three-dimensional textile (3D textile) between the breast prosthesis and the surface on which it rests.

### CROSS-REFERENCE TO RELATED APPLICATION

This application claims priority to German Patent Application Serial No. 20 2005 007 677.5 filed May 17, 2005, which is hereby incorporated by reference in its entirety for all purposes.

### FIELD

The present disclosure relates to a breast prosthesis comprising bodies which copy the shape of the breast, are preferably made from an addition-crosslinking two component silicone rubber mass and which are sealed in plastic films.

### BACKGROUND AND SUMMARY

A method of manufacturing breast prostheses made from dish-shaped bodies which copy the shape of the breast, are made from an addition-crosslinking two component silicone rubber mass and which are sealed in plastic films is known from DE 27 01 627 A for example. The prostheses manufactured by this method copy the natural breast in a near-ideal manner in terms of their appearance and behavior on account of the elasticity, mobility, consistency, and weight of the material used. The breast prostheses are as far as possible secured to the wearer's breast in such a way that they cannot slip. To achieve this, it is known from EP 392 960 A for example to provide the back of breast prostheses of the aforementioned kind with a surrounding raised shoulder within a surrounding lip-shaped edge, this shoulder having secured to it adhesive strips or adhesive pieces which operate in conjunction with the adhesive areas of strips attached to the woman's body with skin-friendly adhesives in such a way that the prosthesis is connected to the holding strips stuck on the skin and can subsequently be detached from these. A Velcro fastening, for example, is provided as a means of securing the prosthesis.

It is known from DE 44 21 516 C1 for example that the abovementioned breast prostheses can be made more comfortable to wear by creating a textile-backed breast prosthesis in which a hollow space between the textile back and the plastic prosthesis can be filled with wadding.

A further development is known from EP 1 232 733 B, in which the admixture of what is called a PCM material to the silicone rubber mass of the breast prosthesis is described. Such a PCM material can, for example, be a paraffin substance with a melting range of approximately 33° C. to 37° C. The paraffins used as a PCM material are paraffins that have been specifically modified for heat-related applications. These paraffins for heat-related applications have a specific heat of fusion or enthalpy of fusion which is very high for organic substances—130 kJ/kg to 150 kJ/kg. If the surface temperature of the skin rises above this temperature level, the PCM material is melted. Melting requires an adequate amount of energy, which is taken as heat from the surface of the skin, this contributing to a pleasant cooling effect.

Further modifications of generic breast prostheses are known; in some of them the breast prosthesis is designed as a multilayer prosthesis, in which the part of the breast prosthesis that is closer to the body is filled with a thixotropic material.

One object of the present disclosure is to further improve the comfort of a breast prosthesis for the wearer.

According to one aspect of the present disclosure, this object is achieved by a breast prosthesis comprising bodies which copy the shape of the breast and made from an addition-crosslinking two component silicone rubber mass and which are sealed in plastic films, and a three-dimensional textile (3D textile) positioned between the breast prosthesis and a surface on which it rests. The three-dimensional textile, also referred to as “3D textile”, may be positioned between the breast prosthesis and the surface on which it rests, i.e. generally the surface of the wearer's body. Such 3D textiles are usually products which are constructed in three layers. They have a top layer and a bottom layer and, between these, a layer of “spacer fibers”. This results in an ultra-light material with an extremely high degree of air distribution, soft cushioning properties, very good shape-regaining characteristics, and good deformability. Firstly, the use of this 3D textile on the back of the prosthesis greatly increases ventilation between the prosthesis and the body, which has a decisive impact on wearer comfort. The soft cushioning properties and good deformability also make the prosthesis feel pleasant to wear.

Further, the 3D textile may contain sweat-absorbing fibers, which can soak up sweat that forms on the skin. In particular, if the intermediate 3D textile layers are designed so as to be detachable, they can be changed regularly, which is particularly advantageous in cases where sweat is absorbed in this way.

The 3D textile may be applied redetachably direct to the back of the breast prosthesis. Detachability can be effected through Velcro strips, double-sided adhesive tape and/or snap-fasteners, or other suitable releasable fastening elements.

The breast prosthesis can have cavities and depressions into which modular cushions comprising a filled chamber may be inserted. These cushions may in principle be filled with materials such as a PCM material for example or, in the simplest case, with water, or with any other liquid. In a modular structure of this kind, the 3D textile is applied detachably or nondetachably direct to the filled chamber.

A further solution provided by the present disclosure is that the breast prosthesis can be inserted into the pocket of a brassiere which has a 3D textile on the side of it that faces the body of the wearer. The protection also relates to such a brassiere which serves to hold breast prostheses of this kind. According to the present disclosure, this brassiere has one or two pockets to hold the breast prostheses, there being a 3D textile on the side facing the body of the wearer. There may also be one or more layers of an additional textile between the 3D textile and the body, such as tulle.

### BRIEF DESCRIPTION OF THE FIGURES

Further details and advantages of the present disclosure are explained with the aid of an exemplary embodiment presented in the drawings.

FIG. 1 shows the structure of a breast prosthesis according to a first exemplary embodiment of the present disclosure;

FIG. 2 shows an alternative structure according to a second embodiment;

FIGS. 3 and 4 show other alternative structures of a breast prosthesis according to the present disclosure;

FIG. 5 shows a brassiere for holding breast prostheses according to an embodiment of the present disclosure; and

FIG. 6 shows a section through the brassiere according to FIG. 5.

### DETAILED DESCRIPTION

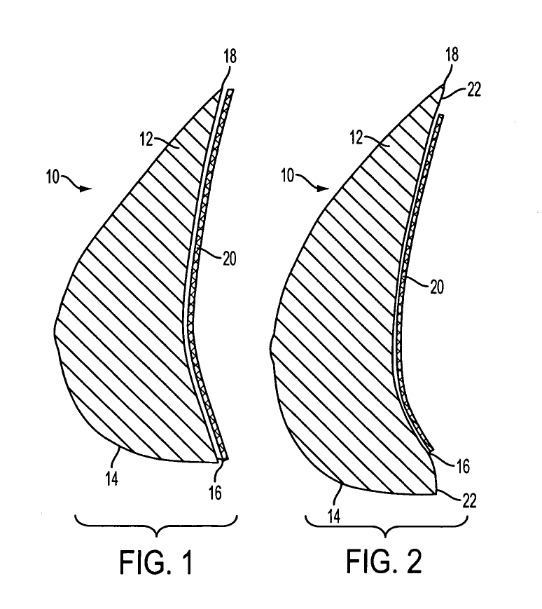

The breast prosthesis 10 presented in FIG. 1 includes a dish-shaped body 12 made from a soft-elastic addition-crosslinking two component rubber mass, the outside of which is covered by a polyurethane film 14 and the inside of which is covered by a polyurethane film 16, these being joined to each other, along a common circumferential edge 18, by a surrounding seam.

On the back of the breast prosthesis in the exemplary embodiment in FIG. 1 there is a 3D textile 20. This 3D textile covers the whole of the back of the breast prosthesis 10.

A 3D textile of this kind is usually constructed in three layers and includes a top layer and a bottom layer with “spacer fibers” between them which determine the thickness of the 3D textile. The thickness of such standard commercial 3D textiles as used in the present solution according to the present disclosure ranges from 1 mm to over 20 mm. Polyester or polyamide fibers are typically used for the 3D textiles. Special sweat-absorbing materials may also be incorporated in the 3D textiles, however.

Known examples of such 3D textiles include “AirX 3D Spacer Fabric” from the company Tytex, “Spacetec” from Heathcoat, “XD-Spacer Fabrics” from Baltex, and “3 mesh” from Müller-Textil. The degree of air circulation between the breast prosthesis 10 and the body of the wearer (not shown here) can be set by selecting a 3D textile of the appropriate thickness. The soft cushioning properties and the good deformability of the 3D textile 20 also mean a high level of wearer comfort.

FIG. 2 is basically the same as FIG. 1, except that the 3D textile is arranged in such a way as to leave a projecting edge 22. The edge may have adhesive pads secured to it to attach the breast prosthesis to the wearer's body.

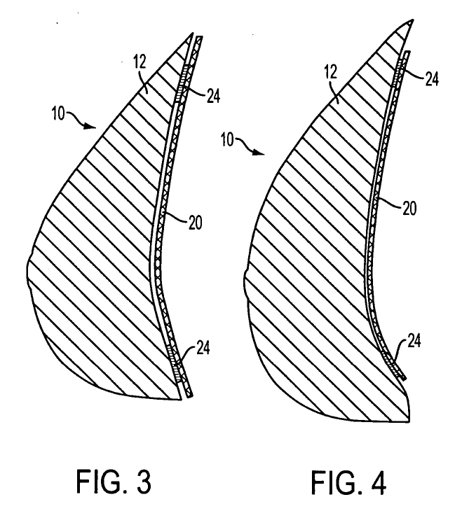

FIGS. 3 and 4 are basically the same as FIGS. 1 and 2, except that they both have a mechanism for attaching the 3D textile 20 to the breast prosthesis 10 in a detachable way. The mechanism in the exemplary embodiment presented here is corresponding Velcro bands 24.

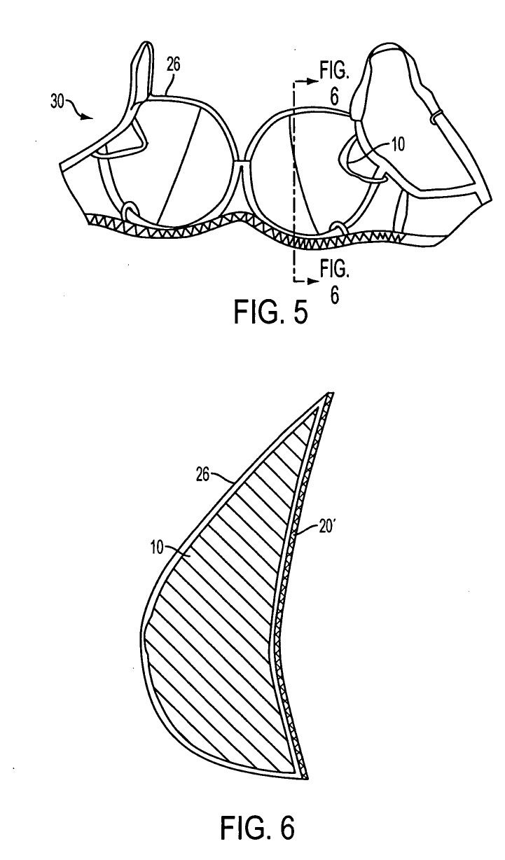

Another variant embodiment of the present disclosure is presented in FIGS. 5 and 6. FIG. 5 shows a brassiere 30 which has pockets to hold prostheses 10. The sectional view along the line 6-6 in FIG. 6 shows the textile pocket 26 of the brassiere 30 with the breast prosthesis 10 inserted into it. The layer 20′ is formed on the back of the pocket 26 detachably or in one piece with the pocket 26 as a 3D textile layer.

1. A breast prosthesis comprising bodies which copy the shape of the breast and a three-dimensional textile (3D textile) positioned between the breast prosthesis and a surface on which it rests.

2. The breast prosthesis as claimed in claim 1, wherein the bodies are made from an addition-crosslinking two component silicone rubber mass and the bodies are sealed in plastic films.

3. The breast prosthesis as claimed in claim 2, wherein the 3D textile contains sweat-absorbing fibers.

4. The breast prosthesis as claimed in claim 2, wherein the 3D textile is applied redetachably direct to a back of the breast prosthesis.

5. The breast prosthesis as claimed in claim 4, wherein the 3D textile is connected to the back of the breast prosthesis by Velcro strips, double-sided adhesive tape and/or snap-fasteners.

6. The breast prosthesis as claimed in claim 2, wherein cushions comprising a filled chamber may be inserted into the breast prosthesis.

7. The breast prosthesis as claimed in claim 6, wherein the breast prosthesis includes cavities and depressions into which the cushions may be inserted.

8. The breast prosthesis as claimed in claim 1, further comprising one or more layers of an additional textile between the 3D textile and the body.

9. The breast prosthesis as claimed in claim 8, wherein said additional textile is tulle.

10. A brassiere for holding a breast prosthesis to be worn by a wearer, comprising one or more pockets to hold the breast prosthesis, wherein at least one of said pockets have a 3D textile on a side facing a body of the wearer.

11. The brassiere of claim 10, wherein the breast prosthesis is shaped to be inserted into one of said pockets of the brassiere.

12. The brassiere of claim 11, wherein the 3D textile contains sweat-absorbing fibers, and wherein the 3D textile is redetachably coupled to the brassiere.

13. A breast prosthesis comprising:
an addition-crosslinking two component silicone rubber mass forming a body in the shape of the breast, the body sealed in plastic films, and
a three-dimensional textile (3D textile) coupled to a wearer-facing surface of the body.

14. The breast prosthesis as claimed in claim 13, wherein the 3D textile is positioned adjacent said surface, where said surface is shaped to be positioned adjacent a wearer.

15. The breast prosthesis as claimed in claim 14, wherein the 3D textile contains sweat-absorbing fibers.

16. The breast prosthesis as claimed in claim 15, wherein the 3D textile is applied redetachably direct to said surface.

17. The breast prosthesis as claimed in claim 16, wherein the 3D textile is connected to the surface by Velcro strips, double-sided adhesive tape and/or snap-fasteners.

18. The breast prosthesis as claimed in claim 13, wherein the breast prosthesis includes cavities and depressions shaped to receive cushions, wherein the cushions comprise a filled chamber.

19. The breast prosthesis as claimed in claim 13, wherein the breast prosthesis includes cavities and depressions shaped to receive cushions, the prosthesis further comprising the cushion, the cushion having a filled chamber.

20. The breast prosthesis as claimed in claim 15, further comprising one or more layers of an additional textile between the 3D textile and the body.
